(19) United States
(12) Patent Application Publication
(10) Pub. No.: US 2002/0038074 A1
(43) Pub. Date: Mar. 28, 2002

(54) Endoscope system having multiaxial-mode laser-light source or substantially producing multiaxial-mode laser light from single-axial-mode laser light

(75) Inventor: Kazuo Hakamata, Kaisei-machi (JP)

Correspondence Address: SUGHRUE, MION, ZINN, MACPEAK & SEAS, PLLC, 2100 Pennsylvania Avenue, N.W., Washington, DC, 20037-3202, US

(73) Assignee: FUJI PHOTO FILM CO., LTD.

(21) Appl. No.: 09/962,250

(22) Filed: Sep. 26, 2001

(30) Foreign Application Priority Data
Sep. 26, 2000 (JP) 292471/2000

(51) Int. Cl. A61B 1/06

(52) U.S. Cl. 600/178

(57) ABSTRACT

In an endoscope system including: a light emission unit emits laser light as illumination light or excitation light; a light guide unit guides the illumination light or the excitation light to an object; and an image pickup unit picks up a normal image formed with reflection light generated by reflection of the illumination light from the object or a fluorescence image emitted from the object in response to the excitation light. The laser light is multiaxial-mode laser light, or the light emission unit includes a plurality of laser-light sources which emit single-axial-mode laser beams having different wavelengths or phases. Alternatively, a vibration unit which vibrates the light guide unit is provided, or a high-frequency signal is superimposed on a driving current of the light emission unit, so that the wavelength of the laser light is shifted among a plurality of values.

### BACKGROUND OF THE INVENTION

1. Field of the Invention

The present invention relates to an endoscope system which illuminates living tissue with illumination light or excitation light, detects reflection of the illumination light from the living tissue or fluorescence light emitted from the living tissue in response to the illumination of the excitation light, and displays an image indicating information on the living tissue.

2. Description of the Related Art

Endoscopes have been widely used for observing internal parts of living bodies, and treating diseased areas of the living bodies while observing the diseased areas. Recently, the following techniques have been proposed for the endoscope systems:

(a) Techniques of illuminating living tissue with illumination light, picking up a normal image formed with reflection light from the living tissue, and displaying the normal image

(b) Techniques of illuminating living tissue with excitation light in a predetermined wavelength range, receiving fluorescence light emitted from fluorescent pigment inherent in the living tissue, and displaying a fluorescence image indicating localization or spread of diseased tissue.

Usually, when living tissue is illuminated with excitation light, normal tissue emits strong fluorescence light as indicated by a solid curve in FIG. 9, and diseased tissue emits only very weak fluorescence light as indicated by a dashed curve in FIG. 9. Therefore, it is possible to determine whether living tissue is normal or diseased by measuring the intensity of the fluorescence light.

When living tissue is illuminated with excitation light in order to display an image based on the intensity of fluorescence light emitted in response to the illumination, the intensity of the excitation light applied to the living tissue is not uniform since the surfaces of the living tissue are uneven. Although the intensity of fluorescence light emitted from the living tissue is proportional to the intensity of the excitation light, the intensity of the excitation light decreases inversely proportional to the square of the distance from the light source. Therefore, fluorescence light received from a diseased area of the living tissue located near to the light source may be stronger than fluorescence light received from a normal area of the living tissue located far from the light source. That is, it is not possible to accurately discriminate properties of the living tissue based on only the intensity of the fluorescence light emitted in response to excitation light.

In order to overcome the above problem, the present inventors have proposed the following methods:

(a) A method of calculating a ratio between intensities of fluorescence light in two different wavelength bands for each pixel, and displaying an image based on the calculated ratio (i.e., a method of displaying an image based on the fact that properties of living tissue are reflected in shapes of fluorescence spectra)

(b) A method of illuminating living tissue with near infrared light as reference light, detecting intensity of near infrared light reflected from the illuminated living tissue, calculating a ratio between intensity of fluorescence light and the reflected near infrared light, and displaying an image based on the calculated ratio, where the near infrared light is equally absorbed by various areas of living tissue having different properties (i.e., a method of displaying an image based on the values in which fluorescence yields are reflected).

In the conventional endoscope systems in which the above methods are used, usually, halogen lamps, xenon lamps, or the like are used as sources of the illumination light, and mercury lamps, xenon lamps, or the like and band-pass filters are used for obtaining the excitation light having a specific wavelength. However, in order to achieve downsizing, energy conservation, cost reduction, and the like, laser-light sources are considered to be used in endoscope systems. Actually, use of a laser-light source as an excitation-light source in an endoscope system has already been proposed.

However, laser-light sources which are conventionally considered to be used in endoscope systems as illumination-light sources or excitation-light sources are laser-light sources which emit single-axial-mode laser light (i.e., monochromatic laser light). Since laser light is coherent, the single-axial-mode illumination light or the single-axial-mode excitation light causes interference and produces an interference pattern. When the illumination light or the excitation light produces an interference pattern, a normal image or a fluorescence image produced by illumination with the illumination light or the excitation light is affected by the interference pattern, and an uneven diagnostic image is obtained. That is, properties of living tissue cannot be accurately indicated in the uneven diagnostic image.

### SUMMARY OF THE INVENTION

An object of the present invention is to provide an endoscope system which uses a laser-light source as a source of illumination light or excitation light, and suppresses unevenness in a diagnostic image.

(1) According to the first aspect of the present invention, there is provided an endoscope system comprising: an illumination-light emission unit including at least one laser-light source which emits multiaxial-mode laser light as illumination light; a light guide unit which guides the illumination light to an object, and illuminates the object with the illumination light; and an image pickup unit which picks up a normal image formed with reflection light which is generated by reflection of the illumination light from the object.

The multiaxial-mode laser light is laser light which includes a plurality of wavelength components. In the endoscope system according to the first aspect of the present invention, any type of laser-light source can be used when the laser-light source emits multiaxial-mode laser light. For example, each of the at least one laser-light source may be a solid-state laser-light source or a semiconductor laser-light source.

Since the illumination-light emission unit in the endoscope system according to the first aspect of the present invention comprises at least one laser-light source which emits multiaxial-mode laser light, interference of the illumination light can be suppressed. The interference of the illumination light causes unevenness in the normal image. Therefore, the unevenness in the normal image can be reduced according to the first aspect of the present invention. Thus, the endoscope system according to the first aspect of the present invention can obtain a clearer diagnostic image, and is more energy-efficient and smaller in size, than the conventional endoscope systems.

The illumination-light emission unit may comprise a plurality of laser-light sources each of which emits multiaxial-mode laser light. In this case, the interference of the illumination light is further suppressed, and the endoscope system can obtain a further clear diagnostic image.

The illumination light may be either monochromatic light or white light. When the illumination light is white light, and the illumination-light emission unit is constituted by a plurality of monochromatic laser-light sources which emit necessary color components of white light, each of the plurality of monochromatic laser-light sources should operate in a multiaxial mode.

(2) According to the second aspect of the present invention, there is provided an endoscope system comprising: an illumination-light emission unit which emits illumination light; a light guide unit which guides the illumination light to an object, and illuminates the object with the illumination light; and an image pickup unit which picks up a normal image formed with reflection light which is generated by reflection of the illumination light from the object. The illumination-light emission unit comprises a plurality of laser-light sources each of which emits single-axial-mode laser light having a wavelength and a phase, and the single-axial-mode laser light emitted from one of the plurality of laser-light sources is different in at least one of the wavelength and the phase from the single-axial-mode laser light emitted from another of the plurality of laser-light sources.

The single-axial-mode laser light is laser light which includes only a single wavelength component.

In the endoscope system according to the second aspect of the present invention, the illumination-light emission unit comprises a plurality of laser-light sources, each of the plurality of laser-light sources emits single-axial-mode laser light, and the single-axial-mode laser light emitted from one of the plurality of laser-light sources is different in at least one of the wavelength and the phase from the single-axial-mode laser light emitted from another of the plurality of laser-light sources. Therefore, it is possible to suppress interference of the illumination light. Since unevenness in the normal image is caused by the interference of the illumination light, the unevenness in the normal image can be reduced according to the second aspect of the present invention. Thus, the endoscope system according to the second aspect of the present invention can obtain a clearer diagnostic image, and is more energy-efficient and smaller in size, than the conventional endoscope systems.

(3) According to the third aspect of the present invention, there is provided an endoscope system comprising: an illumination-light emission unit which emits illumination light; a light guide unit which guides the illumination light to an object, and illuminates the object with the illumination light; an image pickup unit which picks up a normal image formed with reflection light which is generated by reflection of the illumination light from the object; and a vibration unit which vibrates the light guide unit.

In the endoscope system according to the third aspect of the present invention, the light guide unit is vibrated by the vibration unit, and the illumination light is guided through the light guide unit to the object. When the light guide unit is vibrated by the vibration unit, the optical length of the illumination light varies. Therefore, interference of the illumination light applied to the object can be suppressed. Since unevenness in the normal image is caused by the interference of the illumination light, the unevenness in the normal image can be reduced according to the third aspect of the present invention. Thus, the endoscope system according to the third aspect of the present invention can obtain a clearer diagnostic image, and is more energy-efficient and smaller in size, than the conventional endoscope systems.

For example, the light guide unit can be realized by an optical fiber, a lens, and the like. The vibration unit may be realized by any device which varies the optical length of the illumination light by vibrating the light guide unit. In addition, when the light guide unit has a substantial length as in the case of an optical fiber, any portion of the light guide unit may be vibrated.

(4) According to the fourth aspect of the present invention, there is provided an endoscope system comprising: an illumination-light emission unit which emits illumination light; a light guide unit which guides the illumination light to an object, and illuminates the object with the illumination light; and an image pickup unit which picks up a normal image formed with reflection light which is generated by reflection of the illumination light from the object. The illumination-light emission unit comprises a laser-light source which emits as the illumination light laser light having a wavelength, a high-frequency-signal output unit which outputs a high-frequency signal, and a driving-current generation unit which generates a driving current of the laser-light source so that the driving current varies according to the high-frequency signal, and the wavelength of the laser light is shifted among a plurality of values.

The laser-light source may be any laser-light source in which the wavelength of the laser light varies with the driving current.

For example, when the laser-light source is realized by a laser diode, the high-frequency signal can be input into a driving circuit of the laser diode so as to vary the driving current, and thereby the wavelength of the laser light emitted from the laser diode is shifted among the plurality of values according to the high-frequency signal. The shift of the wavelength of the laser light occurs so that the laser light emitted from the laser diode has a wavelength distribution substantially similar to the wavelength distribution in multiaxial-mode laser light.

In addition, the high-frequency signal may have any frequency which can realize the above shift of the wavelength.

In the endoscope system according to the fourth aspect of the present invention, the driving current output to the laser-light source varies with the high-frequency signal supplied from the high-frequency-signal output unit. Therefore, the wavelength of the illumination light emitted from the laser-light source is shifted among a plurality of values according to the high-frequency signal. That is, the illumination light has a wavelength distribution substantially similar to the wavelength distribution in multiaxial-mode laser light. Thus, interference of the illumination light applied to the object can be suppressed. Since unevenness in the normal image is caused by the interference of the illumination light, the unevenness in the normal image can be reduced according to the fourth aspect of the present invention. Consequently, the endoscope system according to the fourth aspect of the present invention can obtain a clearer diagnostic image, and is more energy-efficient and smaller in size, than the conventional endoscope systems.

(5) According to the fifth aspect of the present invention, there is provided an endoscope system comprising: an excitation-light emission unit including at least one laser-light source which emits multiaxial-mode laser light as excitation light; a light guide unit which guides the excitation light to an object, and illuminates the object with the excitation light; and an image pickup unit which picks up a fluorescence image formed with fluorescence light which is emitted from the object in response to illumination with the excitation light.

In the endoscope system according to the fifth aspect of the present invention, any type of laser-light source can be used when the laser-light source emits multiaxial-mode laser light. For example, each of the at least one laser-light source may be a solid-state laser-light source or a semiconductor laser-light source.

Since the excitation-light emission unit in the endoscope system according to the fifth aspect of the present invention comprises at least one laser-light source which emits multiaxial-mode laser light, interference of the excitation light can be suppressed. The interference of the excitation light causes unevenness in the fluorescence image. Therefore, the unevenness in the fluorescence image can be reduced according to the fifth aspect of the present invention. Thus, the endoscope system according to the fifth aspect of the present invention can obtain a clearer diagnostic image, and is more energy-efficient and smaller in size, than the conventional endoscope systems.

The excitation-light emission unit may comprise a plurality of laser-light sources each of which emits multiaxial-mode laser light. In this case, the interference of the excitation light is further suppressed, and the endoscope system can obtain a further clear diagnostic image.

(6) According to the sixth aspect of the present invention, there is provided an endoscope system comprising: an excitation-light emission unit which emits excitation light; a light guide unit which guides the excitation light to an object, and illuminates the object with the excitation light; and an image pickup unit which picks up a fluorescence image formed with fluorescence light which is emitted from the object in response to illumination with the excitation light. The excitation-light emission unit comprises a plurality of laser-light sources each of which emits single-axial-mode laser light having a wavelength and a phase, and the single-axial-mode laser light emitted from one of the plurality of laser-light sources is different in at least one of the wavelength and the phase from the single-axial-mode laser light emitted from another of the plurality of laser-light sources.

In the endoscope system according to the sixth aspect of the present invention, the excitation-light emission unit comprises a plurality of laser-light sources, each of the plurality of laser-light sources emits single-axial-mode laser light, and the single-axial-mode laser light emitted from one of the plurality of laser-light sources is different in at least one of the wavelength and the phase from the single-axial-mode laser light emitted from another of the plurality of laser-light sources. Therefore, it is possible to suppress interference of the excitation light. Since unevenness in the fluorescence image is caused by the interference of the excitation light, the unevenness in the fluorescence image can be reduced according to the sixth aspect of the present invention. Thus, the endoscope system according to the sixth aspect of the present invention can obtain a clearer diagnostic image, and is more energy-efficient and smaller in size, than the conventional endoscope systems.

(7) According to the seventh aspect of the present invention, there is provided an endoscope system comprising: an excitation-light emission unit which emits excitation light; a light guide unit which guides the excitation light to an object, and illuminates the object with the excitation light; an image pickup unit which picks up a fluorescence image formed with fluorescence light which is emitted from the object in response to illumination with the excitation light; and a vibration unit which vibrates the light guide unit.

In the endoscope system according to the seventh aspect of the present invention, the light guide unit is vibrated by the vibration unit, and the excitation light is guided through the light guide unit to the object. When the light guide unit is vibrated by the vibration unit, the optical length of the excitation light varies. Therefore, interference of the excitation light applied to the object can be suppressed. Since the unevenness is caused in the fluorescence image by interference of the excitation light, the unevenness in the fluorescence image can be reduced according to the seventh aspect of the present invention. Consequently, the endoscope system according to the seventh aspect of the present invention can obtain a clearer diagnostic image, and is more energy-efficient and smaller in size, than the conventional endoscope systems.

For example, the light guide unit can be realized by an optical fiber, a lens, and the like. The vibration unit may be realized by any device which varies the optical length of the excitation light by vibrating the light guide unit. In addition, when the light guide unit has a substantial length as in the case of an optical fiber, any portion of the light guide unit may be vibrated.

(8) According to the eighth aspect of the present invention, there is provided an endoscope system comprising: an excitation-light emission unit which emits excitation light; a light guide unit which guides the excitation light to an object, and illuminates the object with the excitation light; and an image pickup unit which picks up a fluorescence image formed with fluorescence light which is emitted from the object in response to illumination with the excitation light. The excitation-light emission unit comprises a laser-light source which emits as the excitation light laser light having a wavelength, a high-frequency-signal output unit which outputs a high-frequency signal, and a driving-current generation unit which generates a driving current of the laser-light source so that the driving current varies according to the high-frequency signal, and the wavelength of the laser light is shifted among a plurality of values.

The laser-light source may be any laser-light source in which the wavelength of the laser light varies with the driving current.

For example, when the laser-light source is realized by a laser diode, the high-frequency signal can be input into a driving circuit of the laser diode so as to vary the driving current, and thereby the wavelength of the laser light emitted from the laser diode is shifted among the plurality of values according to the high-frequency signal. The shift of the wavelength of the laser light occurs so that the laser light emitted from the laser diode has a wavelength distribution substantially similar to the wavelength distribution in multiaxial-mode laser light.

In addition, the high-frequency signal may have any frequency which can realize the above shift of the wavelength.

In the endoscope system according to the eighth aspect of the present invention, the driving current output to the laser-light source varies with the high-frequency signal supplied from the high-frequency-signal output unit. Therefore, the wavelength of the excitation light emitted from the laser-light source is shifted among a plurality of values according to the high-frequency signal. That is, the excitation light has a wavelength distribution substantially similar to the wavelength distribution in multiaxial-mode laser light. Thus, interference of the excitation light applied to the object can be suppressed. Since unevenness in the fluorescence image is caused by the interference of the excitation light, the unevenness in the fluorescence image can be reduced according to the eighth aspect of the present invention. Consequently, the endoscope system according to the eighth aspect of the present invention can obtain a clearer diagnostic image, and is more energy-efficient and smaller in size, than the conventional endoscope systems.

Preferably, in the endoscope systems according to the fifth to eighth aspects of the present invention, the laser-light source is a GaN semiconductor laser element, and the excitation light belongs to a wavelength band within a range of 400 to 420 nm. In this case, the laser-light source can efficiently emit the fluorescence light.

### DESCRIPTION OF THE DRAWINGS

FIG. 1 is a diagram illustrating an outline of a construction of a fluorescence endoscope system as a first embodiment of the present invention.

FIG. 2 is a diagram illustrating an example of the optical transmission filter.

FIG. 3 is a diagram illustrating an example of a spectrum of multiaxial-mode laser light.

FIG. 4 is a diagram illustrating an outline of a construction of a fluorescence endoscope system as a second embodiment of the present invention.

FIG. 5 is a diagram illustrating an example of a spectrum of single-axial-mode laser light.

FIG. 6 is a diagram illustrating an outline of a construction of a fluorescence endoscope system as a third embodiment of the present invention.

FIG. 7 is a diagram illustrating an outline of a construction of a fluorescence endoscope system as a fourth embodiment of the present invention.

FIG. 8 is a graph indicating a relationship between a driving current of a (GaN) semiconductor laser element and the wavelength of laser light emitted from the semiconductor laser element.

FIG. 9 is a graph indicating spectra of fluorescence light when normal tissue and diseased tissue are illuminated with excitation light.

### DESCRIPTION OF PREFERRED EMBODIMENTS

Embodiments of the present invention are explained in detail below with reference to drawings.

Construction of First Embodiment

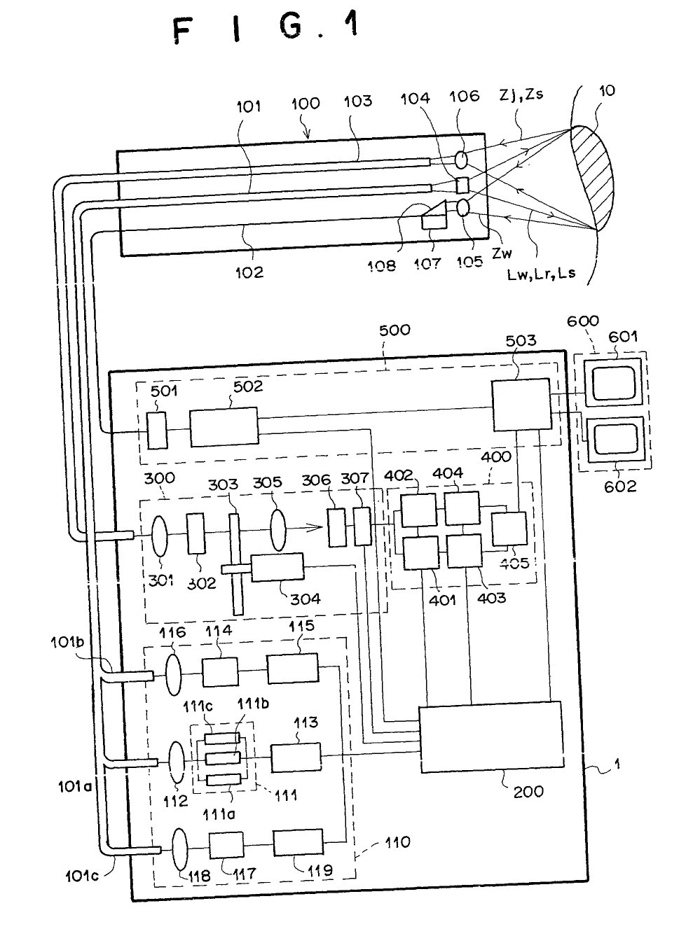

The first embodiment of the present invention is explained below. FIG. 1 is a diagram illustrating an outline of a construction of the fluorescence endoscope system as the first embodiment of the present invention. The fluorescence endoscope system of FIG. 1 realizes the functions of both the first and fifth aspects of the present invention. The fluorescence endoscope system of FIG. 1 comprises an image-information processing unit 1, an endoscope insertion unit 100, and a monitor unit 600. The endoscope insertion unit 100 is inserted into a portion of interest in a body of a patient. The image information processing unit 1 processes image information obtained from living tissue 10, and outputs processed image information. The monitor unit 600 displays a visible image based on the image information processed by the image information processing unit 1.

Specifically, the image information processing unit 1 comprises an illumination unit 110, an image detection unit 300, an image calculation unit 400, a display-signal processing unit 500, and a computer 200.

The illumination unit 110 comprises three light sources which emit white light Lw, excitation light Lr, and reference light Ls, respectively. The image detection unit 300 picks up an autofluorescence image Zj and EL reflection image Zs, converts the images Zj and Zs into digital values, and outputs the digital values as two-dimensional image data. The autofluorescence image Zj is carried by autofluorescence light which is emitted from the living tissue 10 in response to illumination with the excitation light Lr, and the reflection image Zs is carried by a portion of the reference light Ls reflected from the living tissue 10 when the living tissue 10 is illuminated with the reference light Ls.

The image calculation unit 400 performs calculations on the two-dimensional image data of the autofluorescence image Zj, for example, for distance correction, and assigns colors to the calculated values of the autofluorescence image Zj for color indication of the autofluorescence image Zj. In addition, the image calculation unit 400 assigns brightness levels to the two-dimensional image data of the reflection image Zs for displaying the reflection image Zs, synthesizes the image information for the color indication of the autofluorescence image Zj and the image information for displaying the reflection image Zs, and outputs the synthesized image information.

The display-signal processing unit 500 receives an image signal of a normal image Zw from the normal-image pickup element 107, and converts the normal image Zw into digital values as two-dimensional image data, where the normal image Zw is obtained as a reflection image when the living tissue 10 is illuminated with the white light Lw. In addition, the display-signal processing unit 500 converts into video signals the two-dimensional image data of the normal image Zw and the image information output from the image calculation unit 400, and outputs the video signals to the monitor unit 600. The computer 200 controls the respective units in the fluorescence endoscope system.

The endoscope insertion unit 100 comprises a light guide 101, a CCD cable 102, an image fiber 103, an illumination lens 104, an objective lens 105, a condenser lens 106, a normal-image pickup element 107, and a reflection prism 108.

The image fiber 103 is a quartz-glass fiber. The light guide 101, the CCD cable 102, and the image fiber 103 extend through the endoscope insertion unit 100. At the front end of the endoscope insertion unit 100, the illumination lens 104 is coupled to the front end of the light guide 101, the objective lens 105 is coupled to the front end of the CCD cable 102, and the condenser lens 106 is coupled to the front end of the image fiber 103. The prism 108 is coupled to the normal-image pickup element 107, and the normal-image pickup element 107 and the prism 108 are arranged at the front end of the CCD cable 102.

The light guide 101 is made of a white-light guide 101a, an excitation-light guide 101b, a reference-light guide 101c, which are bundled together so as to form a cable. Each of the white-light guide 111a and the reference-light guide 101c is made of a multi-component glass fiber, and the excitation-light guide 101b is made of a quartz-glass fiber. The rear ends of the white-light guide 101a, the excitation-light guide 101b, and the reference-light guide 101c are each connected to the illumination unit 110. Further, the rear end of the CCD cable 102 is connected to the display-signal processing unit 500, and the rear end of the image fiber 103 is connected to the image detection unit 300.

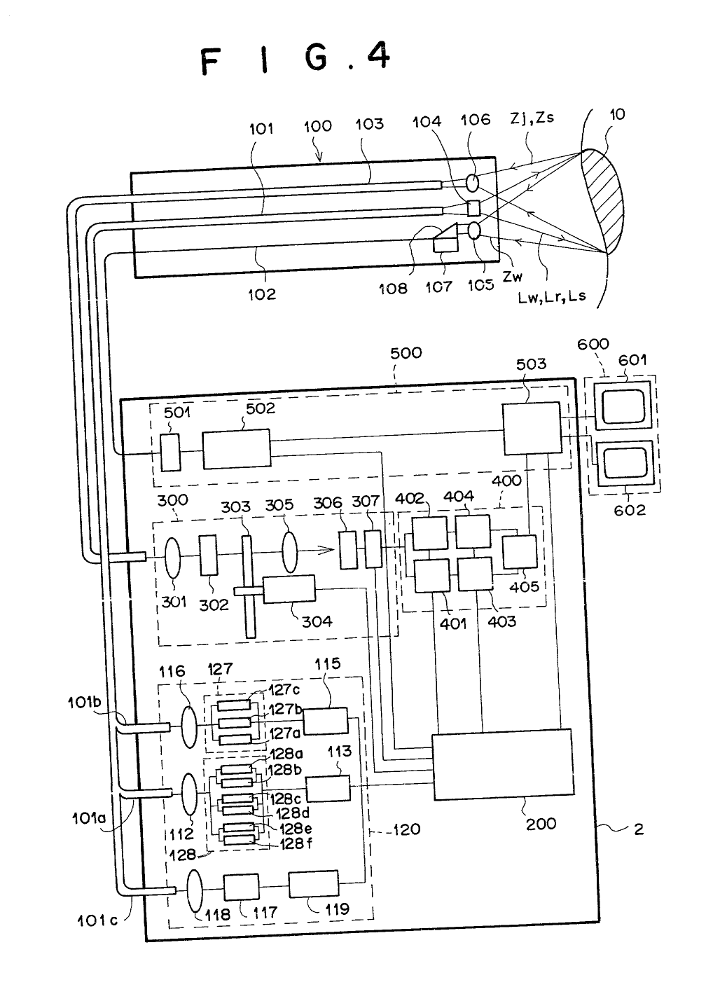

The illumination unit 110 comprises a white-light source 111, a white-light condenser lens 112, and a power supply 113. The white-light source 111 emits the white light Lw for obtaining the normal image Zw. The white-light condenser lens 112 collects the white light Lw emitted from the white-light source 111. The power supply 113 is electrically connected to the white-light source 111, and supplies electric power to the white-light source 111. The white-light source 111 comprises a red semiconductor laser element 111a, a green semiconductor laser element 111b, and a blue semiconductor laser element 111c. Each of these semiconductor laser elements 111a, 111b, and 111c emits multiaxial-mode laser light. The multiaxial-mode laser light is laser light which has a spectral distribution including a plurality of wavelengths λ, for example, as illustrated in FIG. 3, and causes little interference.

The illumination unit 110 also comprises a GaN semiconductor laser element 114, a power supply 115, and a condenser lens 116. The GaN semiconductor laser element 114 emits multiaxial-mode laser light as the excitation light Lr for obtaining the autofluorescence image Zj. The multiaxial-mode laser light emitted from the GaN semiconductor laser element 114 also causes little interference. The power supply 115 is electrically connected to the GaN semiconductor laser element 114, and supplies electric power to the GaN semiconductor laser element 114. The condenser lens 116 collects the excitation light Lr emitted from the GaN semiconductor laser element 114.

The illumination unit 110 further comprises a reference-light source 117, a condenser lens 118, and a power supply 119. The reference-light source 117 emits the reference light Ls for obtaining the reflection image Zs. For example, the reference-light source 117 is an SLD (superluminescent diode) which emits light belonging to a predetermined wavelength band in the infrared wavelength range, having a short coherence length, and causing little interference. The condenser lens 118 collects the reference light Ls emitted from the reference-light source 117. The power supply 119 is electrically connected to the reference-light source 117, and supplies electric power to the reference-light source 117.

The image detection unit 300 is connected to the image fiber 103, and comprises a collimator lens 301, an excitation-light cut filter 302, an optical transmission filter 303, a filter rotation device 304, an autofluorescence-light condenser lens 305, a high-sensitivity autofluorescence-image pickup element 306, and an analog-to-digital converter 307.

The collimator lens 301 guides the autofluorescence image Zj or the reflection image Zs to an image forming system after the autofluorescence image Zj or the reflection image Zs is transmitted through the image fiber 103. The excitation-light cut filter 302 cuts off wavelength components in the vicinity of the wavelengths of the excitation light Ls. The optical transmission filter 303 cuts out a desired wavelength component of the autofluorescence image Zj or the reflection image Zs which has passed through the excitation-light cut filter 302. The filter rotation device 304 rotates the optical transmission filter 303. The autofluorescence-light condenser lens 305 is provided for forming the autofluorescence image Zj or the reflection image Zs. The high-sensitivity autofluorescence-image pickup element 306 picks up the autofluorescence image Zj or the reflection image Zs formed by the autofluorescence-light condenser lens 305. The analog-to-digital converter 307 converts the autofluorescence image Zj or the reflection image Zs picked up by the high-sensitivity autofluorescence-image pickup element 306, into the aforementioned digital values as the two-dimensional image data.

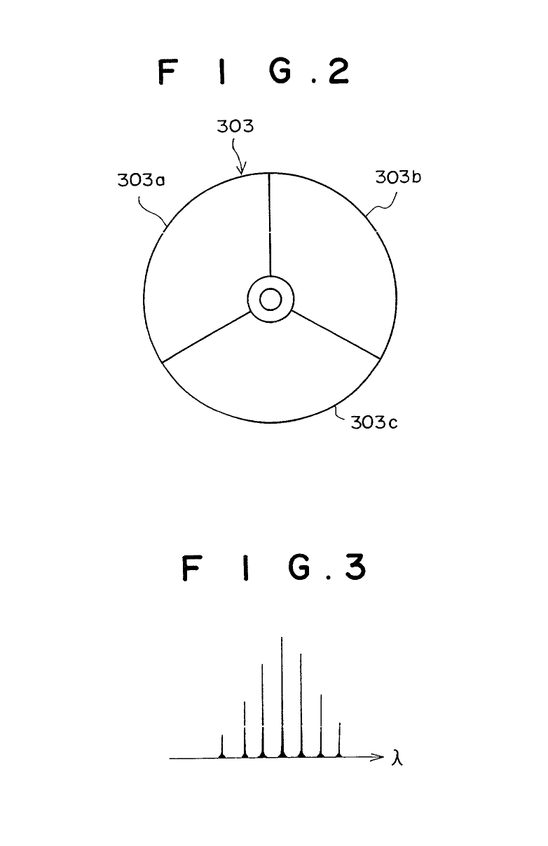

FIG. 2 is a diagram illustrating an example of the optical transmission filter 303. The optical transmission filter 303 comprises three types of band-pass filters 303a, 303b, and 303c. The band-pass filter 303a allows passage of a broadband autofluorescence image in the wavelength range of 430 to 730 nm, the band-pass filter 303b allows passage of a narrowband autofluorescence image in the wavelength range of 430 to 530 nm, and the band-pass filter 303c allows passage of a reflection image Zs in the wavelength range of 750 to 900 nm.

The image calculation unit 400 comprises an autofluorescence-image memory 401, a reflection-image memory 402, an autofluorescence-image calculation unit 403, a reflection-image calculation unit 404, and an image synthesis unit 405. The autofluorescence-image memory 401 stores digitized data of autofluorescence images in two different wavelength bands. The reflection-image memory 402 stores data representing a reflection image. The autofluorescence-image calculation unit 403 performs calculation based on ratios between corresponding pixel values of the autofluorescence images in the two different wavelength bands, and assigns colors to values obtained by the calculation for the respective pixels so as to produce data of a color image. The reflection-image calculation unit 404 assigns brightness levels to the respective pixel values in the data stored in the reflection-image memory 402 so as to produce data of a brightness image. The image synthesis unit 405 synthesizes the data of the color image (obtained in the autofluorescence-image calculation unit 403) and the data of the brightness image (obtained in from the reflection-image calculation unit 404) so as to produce data of a synthesized image, and outputs the data of the synthesized image.

The display-signal processing unit 500 comprises an analog-to-digital converter 501, a normal-image memory 502, and a video-signal processing circuit 503. The analog-to-digital converter 501 digitizes the image signal of the normal image Zw, which is output from the normal-image pickup element 107, so as to generate the aforementioned two-dimensional image data of the normal image Zw. The normal-image memory 502 stores the digitized image data (two-dimensional image data) of the normal image Zw. The video-signal processing circuit 503 converts the two-dimensional image data of the normal image Zw (output from the normal-image memory 502) and the synthesized image (output from the image synthesis unit 405) into video signals.

The monitor unit 600 comprises a monitor 601 for displaying the normal image Zw and a monitor 602 for displaying the synthesized image.

Operation of First Embodiment

The operations of the fluorescence endoscope system as the first embodiment of the present invention are explained below for the case where a synthesized image is displayed by using digitized data of autofluorescence images in two different wavelength bands and a reflection image.

First, in order to obtain the autofluorescence images in the two different wavelength bands, the computer 200 activates the power supply 115 by sending a control signal to the power supply 115. Then, the GaN semiconductor laser element 114 emits multiaxial-mode excitation light Lr having a center wavelength of 410 nm. The excitation light Lr is collected by the condenser lens 116, and enters the excitation-light guide 101b. The excitation light Lr is guided through the excitation-light guide 101b to the front end of the endoscope insertion unit 100, and is then applied to the living tissue 10 through the illumination lens 104.

When the living tissue 10 is illuminated with the excitation light Lr, autofluorescence light carrying an autofluorescence image Zj is emitted from the living tissue 10. The autofluorescence light is collected by the condenser lens 106, and enters the front end of the image fiber 103. Then, the autofluorescence light is guided through the image fiber 103 and the collimator lens 301 to the excitation-light cut filter 302. The autofluorescence light passes through the excitation-light cut filter 302, and is incident on the optical transmission filter 303. The excitation-light cut filter 302 is a long-wave pass filter which allows passage of any fluorescence light having a wavelength equal to or above 420 nm. Since the center wavelength of the excitation light Lr is 410 nm, a portion of the excitation light Lr which is reflected by the living tissue 10 is cut off by the excitation-light cut filter 302, i.e., does not enter the optical transmission filter 303.

At this time, the optical transmission filter 303 is rotated by the filter rotation device 304 under control of the computer 200 so that the autofluorescence light is incident on the band-pass filter 303a. Then, a portion of the autofluorescence light carrying a broadband autofluorescence image passes through the band-pass filter 303a, and the broadband autofluorescence image is formed by the autofluorescence-light condenser lens 305 and picked up by the high-sensitivity autofluorescence-image pickup element 306. The high-sensitivity autofluorescence-image pickup element 306 outputs an image signal representing the broadband autofluorescence image to the analog-to-digital converter 307, which digitizes the image signal. The digitized image signal is stored in a broadband-autofluorescence-image area (not shown) in the autofluorescence-image memory 401.

Next, the optical transmission filter 303 is rotated by the filter rotation device 304 under control of the computer 200 so that the autofluorescence light is incident on the band-pass filter 303b. Then, a portion of the autofluorescence light carrying a narrowband autofluorescence image passes through the band-pass filter 303b, and the narrowband autofluorescence image is formed by the autofluorescence-light condenser lens 305 and picked up by the high-sensitivity autofluorescence-image pickup element 306. The high-sensitivity autofluorescence-image pickup element 306 outputs an image signal representing the narrowband autofluorescence image to the analog-to-digital converter 307, which digitizes the image signal. The digitized image signal is stored in a narrowband-autofluorescence-image area (not shown) in the autofluorescence-image memory 401.

Thereafter, in order to obtain the reflection image Zs, the computer 200 activates the power supply 119 by sending a control signal to the power supply 119. Then, the reference-light source 117 emits reference light Ls having a center wavelength at a predetermined infrared wavelength. The reference light Ls is collected by the condenser lens 118, and enters the reference-light guide 101c. The reference light Ls is guided through the reference-light guide 110c to the front end of the endoscope insertion unit 100, and is then applied through the illumination lens 104 to the living tissue 10.

When the living tissue 10 is illuminated with the reference light Ls, a portion of the reference light Ls carrying a reflection image Zs is reflected as reflection light from the living tissue 10. The reflection light is collected by the condenser lens 106, and enters the front end of the image fiber 103. Then, the autofluorescence light is guided through the image fiber 103 and the collimator lens 301 to the excitation-light cut filter 302. The reflection light passes through the excitation-light cut filter 302, and is incident on the optical transmission filter 303.

At this time, the optical transmission filter 303 is rotated by the filter rotation device 304 under control of the computer 200 so that the reflection light is incident on the band-pass filter 303c. The band-pass filter 303c allows passage of the reflection light carrying the reflection image Zs. Thus, the reflection image Zs carried by the reflection light is formed by the autofluorescence-light condenser lens 305, and picked up by the high-sensitivity autofluorescence-image pickup element 306. An image signal representing the reflection image Zs is output from the high-sensitivity autofluorescence-image pickup element 306 to the analog-to-digital converter 307, digitized by the analog-to-digital converter 307, and stored in the reflection-image memory 402.

The autofluorescence-image calculation unit 403 performs calculation based on ratios between corresponding pixel values of the broadband and narrowband autofluorescence images stored in the autofluorescence-image memory 401, and assigns colors to values obtained by the calculation for the respective pixels so as to generate a color image signal for color indication of the autofluorescence images. In addition, the reflection-image calculation unit 404 assigns brightness levels to the data of the reflection image Zs stored in the reflection-image memory 402 so as to generate a brightness image signal for displaying the reflection image Zs. Then, the image synthesis unit 405 synthesizes the color image signal and the brightness image signal so as to generate a synthesized image signal, which is then output to the video-signal processing circuit 503. The video-signal processing circuit 503 converts the synthesized image signal into a digital. signal, and supplies the digital signal to the monitor unit 600 so that a synthesized image is displayed by the monitor 602.

On the other hand, in order to display the normal. image Zw, the computer 200 activates the power supply 113 by sending a control signal to the power supply 113. Then, red, green, and blue multiaxial-mode laser light beams are emitted from the red, green, and blue semiconductor laser elements 111a, 111b, and 111c, and synthesized into the white light Lw. The white light Lw is collected by the white-light condenser lens 112, and guided through the white-light guide 101a to the front end of the endoscope insertion unit 100. Then, the white light Lw is applied through the illumination lens 104 to the living tissue 10. A portion of the white light Lw is reflected as reflection light from the living tissue 10, collected by the objective lens 105, and reflected by the prism 108 so as to form the normal image Zw on the normal-image pickup element 107. The normal-image pickup element 107 picks up the normal image Zw, and outputs an image signal representing the normal image Zw. The image signal output from the normal-image pickup element 107 is transmitted through the CCD cable 102 to the display-signal processing unit 500, and enters the analog-to-digital converter 501. Next, the image signal is digitized in the analog-to-digital converter 501, and the digitized image signal is stored in the normal-image memory 502. Thereafter, the digitized image signal of the normal image Zw, which is stored in the normal-image memory 502, is converted into an analog video signal by the video-signal processing circuit 503, and the video signal is supplied to the monitor unit 600. Thus, the normal image Zw is displayed by the monitor 601.

The above operations for calculating and displaying the synthesized image and the normal image Zw are controlled by the computer 200.

As explained above, the illumination unit 110 in the fluorescence endoscope system as the first embodiment of the present invention comprises laser-light sources for emitting multiaxial-mode white light and multiaxial-mode excitation light. Therefore, it is possible to suppress interference of each of the white light and the excitation light. Since unevenness in the normal image is caused by the interference of the white light, and unevenness in the autofluorescence image is caused by the interference of the excitation light. Thus, the unevenness in the normal image and the autofluorescence image can be reduced. Consequently, the fluorescence endoscope system as the first embodiment of the present invention can obtain a clearer diagnostic image, and is more energy-efficient and smaller in size, than the conventional endoscope systems.

Second Embodiment

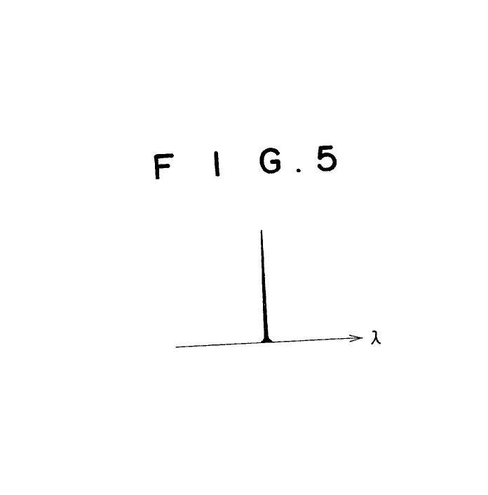

The second embodiment of the present invention is explained below. FIG. 4 is a diagram illustrating an outline of a construction of the fluorescence endoscope system as the second embodiment of the present invention. In FIG. 4, elements having the same reference numbers as FIG. 1 have the same functions as the corresponding elements in FIG. 1, and explanations of the functions of the common elements are not repeated below. The fluorescence endoscope system of FIG. 4 realizes the functions of both the second and sixth aspects of the present invention. The fluorescence endoscope system of FIG. 4 is different from the fluorescence endoscope system of FIG. 1 in the light sources of the white light Lw and the excitation light Lr. That is, the fluorescence endoscope system of FIG. 4 comprises an illumination unit 120 instead of the illumination unit 110.

The illumination unit 120 comprises a white-light source 128 instead of the white-light source 111, and an excitation-light source 127 instead of the GaN semiconductor laser element 114.

The white-light source 128 comprises two red semiconductor laser elements 128a and 128b, two green semiconductor laser elements 128c and 128d, and two blue semiconductor laser elements 128e and 128f. Each of the semiconductor laser elements 128a, 128b, 128c, 128d, 128e, and 128f emits a single-axial-mode laser light beam. The single-axial-mode laser light beam is a laser light beam which has a spectral distribution including a single wavelength 1, for example, as illustrated in FIG. 5. In addition, the wavelengths of the red laser light beams emitted from the red semiconductor laser elements 128a and 128b are different, the wavelengths of the green laser light beams emitted from the green semiconductor laser elements 128c and 128d are different, and the wavelengths of the blue laser light beams emitted from the blue semiconductor laser elements 128e and 128f are different.

The excitation-light source 127 comprises three GaN semiconductor laser elements 127a, 127b, and 127c. Each of the three GaN semiconductor laser elements 127a, 127b, and 127c emits a single-axial-mode laser light beam which constitutes the excitation light Lr, and the wavelengths of the single-axial-mode laser light beams emitted from the three GaN semiconductor laser elements 127a, 127b, and 127c are in the vicinity of 410 nm, and different from each other.

When the white-light source 128 and the excitation-light source 127 in the illumination unit 120 are constructed as above, interference of each of the white light Lw and the excitation light Lr can be suppressed. Except for the white-light source 128 and the excitation-light source 127, the fluorescence endoscope system as the second embodiment of the present invention operates in a similar manner to the fluorescence endoscope system as the first embodiment of the present invention.

As explained above, in the illumination unit 120 in the fluorescence endoscope system as the second embodiment of the present invention, the white-light source comprises more than one laser-light source for each color component of the white light Lw, and the excitation-light source also comprises more than one laser-light source, where each of the more than one laser-light source provided for each color component in the white-light source emits single-axial-mode laser light having a different wavelength, and each of the more than one laser-light source in the excitation-light source emits single-axial-mode laser light having a different wavelength. Therefore, it is possible to suppress interference of each of the white light and the excitation light. Since unevenness in the normal image is caused by interference of the white light, and unevenness in the autofluorescence image is caused by interference of the excitation light, the unevenness in the normal image and the autofluorescence image can be reduced. Thus, the fluorescence endoscope system as the second embodiment of the present invention can obtain a clearer diagnostic image, and is more energy-efficient and smaller in size, than the conventional endoscope systems.

Third Embodiment

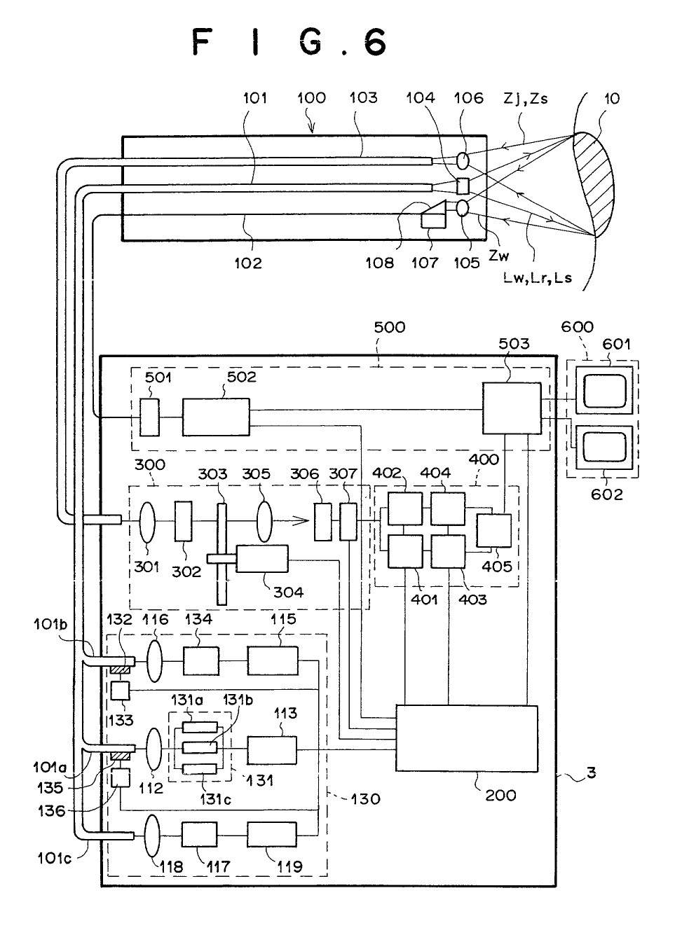

The third embodiment of the present invention is explained below. FIG. 6 is a diagram illustrating an outline of a construction of the fluorescence endoscope system as the third embodiment of the present invention. In FIG. 6, elements having the same reference numbers as FIG. 1 have the same functions as the corresponding elements in FIG. 1, and explanations of the functions of the common elements are not repeated below. The fluorescence endoscope system of FIG. 6 realizes the functions of both the third and seventh aspects of the present invention.

The fluorescence endoscope system of FIG. 6 is different from the fluorescence endoscope system of FIG. 1 in that a vibrator (or shaker) 135 is attached to the white-light guide 101a, a vibrator (or shaker) 132 is attached to the excitation-light guide 101b, and controllers 136 and 133 for the vibrators 135 and 132 are provided. In addition, the fluorescence endoscope system of FIG. 6 comprises a white-light source 131 instead of the white-light source 111, and the white-light source 131 comprises a red semiconductor laser element 131a, a green semiconductor laser element 131b, and a blue semiconductor laser element 131c. Each of these semiconductor laser elements 131a, 131b, and 131c emits single-axial-mode laser light. Further, the fluorescence endoscope system of FIG. 6 comprises a GaN semiconductor laser element 134 instead of the GaN semiconductor laser element 114, and the GaN semiconductor laser element 134 emits single-axial-mode laser light having a wavelength of 410 nm.

The vibrator 135 is arranged near the entrance port of the white-light guide 101a so as to vibrate the white-light guide 101a, where the white light Lw emitted from the white-light source 131 enters the white-light guide 101a through the entrance port. The controller 136 is electrically connected to the vibrator 135, and controls the vibrator 135 so as to activate the vibrator 135 in synchronization with the emission of the white light Lw from the white-light source 131.

In addition, the vibrator 132 is arranged near the entrance port of the white-light guide 101b so as to vibrate the excitation-light guide 101b, where the excitation light Lr emitted from the GaN semiconductor laser element 134 enters the excitation-light guide 101b through the entrance port. The controller 133 is electrically connected to the vibrator 132, and controls the vibrator 132 so as to activate the vibrator 132 in synchronization with the emission of the excitation light Lr from the GaN semiconductor laser element 134.

According to the above construction, the white-light guide 101a is vibrated by the vibrator 135, and the white light Lw is guided through the white-light guide 110a to the front end of the endoscope insertion unit 100. In addition, the excitation-light guide 101b is vibrated by the vibrator 132, and the excitation light Lr is guided through the excitation-light guide 101b to the front end of the endoscope insertion unit 100. When the white-light guide 101a and the excitation-light guide 101b are vibrated by using the vibrators 135 and 132, the optical lengths of the white light Lw and the excitation light Lr vary. Therefore, interference of each of the white light Lw and the excitation light Lr, which are applied to the living tissue 10, can be suppressed. Since unevenness in the normal image is caused by the interference of the white light, and unevenness in the autofluorescence image is caused by the interference of the excitation light, the unevenness in the normal image and the autofluorescence image can be reduced. Thus, the fluorescence endoscope system as the third embodiment of the present invention can obtain a clearer diagnostic image, and is more energy-efficient and smaller in size, than the conventional endoscope systems.

Except for the above constructions for emitting and guiding the white light Lw and the excitation light Lr, the fluorescence endoscope system as the third embodiment of the present invention operates in a similar manner to the fluorescence endoscope system as the first embodiment of the present invention.

Fourth Embodiment

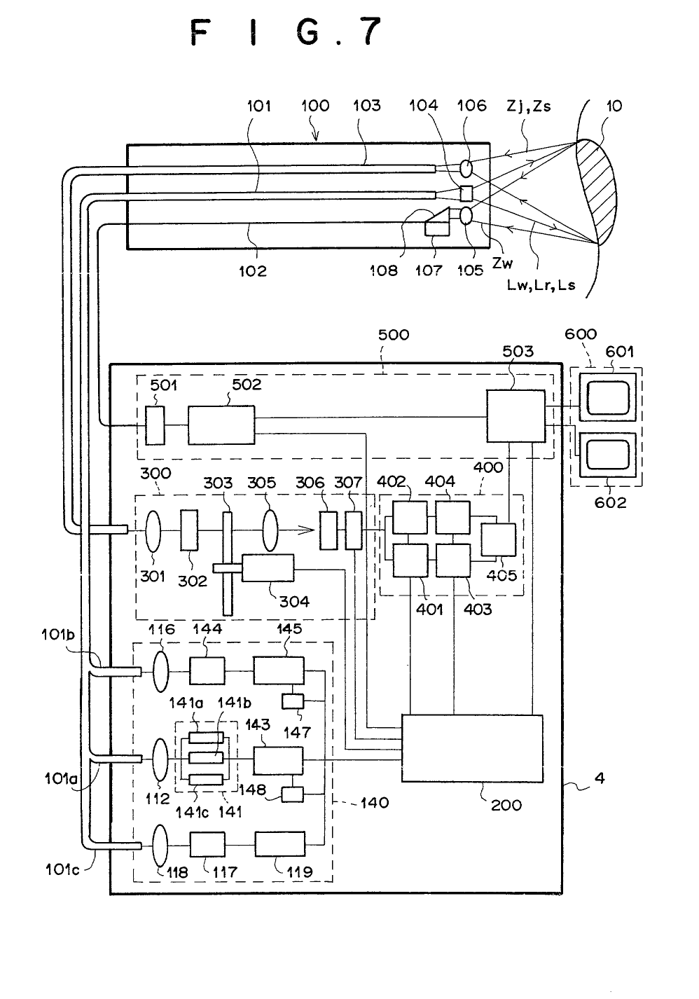

The fourth embodiment of the present invention is explained below. FIG. 7 is a diagram illustrating an outline of a construction of the fluorescence endoscope system as the fourth embodiment of the present invention. In FIG. 7, elements having the same reference numbers as FIG. 1 have the same functions as the corresponding elements in FIG. 1, and explanations of the functions of the common elements are not repeated below. The fluorescence endoscope system of FIG. 7 realizes the functions of both the fourth and eighth aspects of the present invention. The fluorescence endoscope system of FIG. 7 is different from the fluorescence endoscope system of FIG. 1 in the construction of the illumination unit 140.

In the illumination unit 140, a white-light source 141 is provided instead of the white-light source 111, and the white-light source 141 comprises a red semiconductor laser element 141a, a green semiconductor laser element 141b, and a blue semiconductor laser element 141c. Each of the semiconductor laser elements 141a, 141b, and 141c emits single-axial-mode laser light. In addition, a GaN semiconductor laser element 144 is provided instead of the GaN semiconductor laser element 114, and the GaN semiconductor laser element 144 emits single-axial-mode laser light having a wavelength of 410 nm.

Further, a power supply 143 is provided instead of the power supply 113, and a high-frequency-signal output unit 148 is connected to the power supply 143. The high-frequency-signal output unit 148 supplies a high-frequency signal to the power supply 143, and the high-frequency signal is superimposed on an original driving current generated by the power supply 143 per se so that the driving current output from the power supply 143 varies with the high-frequency signal.

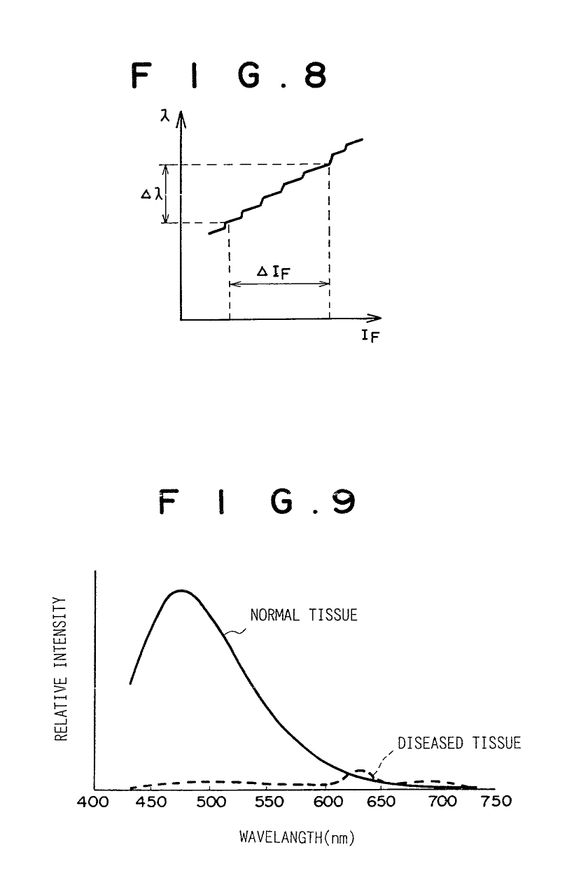

FIG. 8 is a diagram indicating a relationship between a driving current of a semiconductor laser element and the wavelength of laser light emitted from the semiconductor laser element. As illustrated in FIG. 8, the variation Δ I_{F} in the driving current causes a variation Δλ in the wavelength of the laser light. In addition, the wavelength of the laser light varies stepwise with the variation in the driving current.

Therefore, the white light Lw emitted from the white-light source 141 has a wavelength distribution similar to the wavelength distribution of multiaxial-mode laser light.

Similarly, a power supply 145 is provided instead of the power supply 115, and a high-frequency-signal output unit 147 is connected to the power supply 145. The high-frequency-signal output unit 147 supplies a high-frequency signal to the power supply 145, and the high-frequency signal is superimposed on an original driving current generated by the power supply 145 per se so that the driving current output from the power supply 145 varies with the high-frequency signal. Thus, the variation in the driving current causes a variation in the wavelength of the excitation light Lr. That is, the GaN semiconductor laser element 144 emits the excitation light Lr which has a wavelength distribution similar to the wavelength distribution of multiaxial-mode laser light. That is, the excitation light Lr has substantially a plurality of different wavelengths.

According to the above construction, the driving current output from the power supply 143 to the white-light source 141 varies with the high-frequency signal supplied from the high-frequency-signal output unit 148, and the driving current output from the power supply 145 to the GaN semiconductor laser element 144 varies with the high-frequency signal supplied from the high-frequency-signal output unit 147. Therefore, the wavelength of each color component of the white light Lw emitted from the white-light source 141 is shifted among a plurality of values, and the wavelength of the excitation light Lr emitted from the GaN semiconductor laser element 144 is also shifted among a plurality of values. That is, each of the white light Lw and the excitation light Lr has a wavelength distribution similar to the wavelength distribution of multiaxial-mode laser light. Thus, interference of each of the white light Lw and the excitation light Lr, which are applied to the living tissue 10, can be suppressed. Since unevenness in the normal image is caused by the interference of the white light, and unevenness in the autofluorescence image is caused by the interference of the excitation light, the unevenness in the normal image and the autofluorescence image can be reduced. Consequently, the fluorescence endoscope system as the fourth embodiment of the present invention can obtain a clearer diagnostic image, and is more energy-efficient and smaller in size, than the conventional endoscope systems.

Except for the above constructions for emitting the white light Lw and the excitation light Lr, the fluorescence endoscope system as the fourth embodiment of the present invention operates in a similar manner to the fluorescence endoscope system as the first embodiment of the present invention.

Other Matters

(i) In the first to fourth embodiments, the center wavelengths of the excitation-light sources can be chosen in the range of about 400 to 420 nm.

(ii) Although, in the first to fourth embodiments, the normal image Zw and the synthesized image are displayed in the two monitors, the normal image Zw and the synthesized image may be alternately displayed by a single monitor.

What is claimed is:

1. An endoscope system comprising:
an illumination-light emission unit including at least one laser-light source which emits multiaxial-mode laser light as illumination light;
a light guide unit which guides said illumination light to an object, and illuminates the object with the illumination light; and
an image pickup unit which picks up a normal image formed with reflection light which is generated by reflection of said illumination light from said object.

2. An endoscope system according to claim 1, wherein said illumination-light emission unit comprises a plurality of laser-light sources each of which emits multiaxial-mode laser light.

3. An endoscope system comprising:
an illumination-light emission unit which emits illumination light;
a light guide unit which guides said illumination light to an object, and illuminates the object with the illumination light; and
an image pickup unit which picks up a normal image formed with reflection light which is generated by reflection of said illumination light from said object;
said illumination-light emission unit comprises a plurality of laser-light sources each of which emits single-axial-mode laser light having a wavelength and a phase, and the single-axial-mode laser light emitted from one of the plurality of laser-light sources is different in at least one of the wavelength and the phase from the single-axial-mode laser light emitted from another of the plurality of laser-light sources.

4. An endoscope system comprising:
an illumination-light emission unit which emits illumination light;
a light guide unit which guides said illumination light to an object, and illuminates the object with the illumination light;
an image pickup unit which picks up a normal image formed with reflection light which is generated by reflection of said illumination light from said object; and
a vibration unit which vibrates said light guide unit.

5. An endoscope system comprising:
an illumination-light emission unit which emits illumination light;
a light guide unit which guides said illumination light to an object, and illuminates the object with the illumination light; and
an image pickup unit which picks up a normal image formed with reflection light which is generated by reflection of said illumination light from said object;
said illumination-light emission unit comprises,
a laser-light source which emits as said illumination light laser light having a wavelength,
a high-frequency-signal output unit which outputs a high-frequency signal, and
a driving-current generation unit which generates a driving current of said laser-light source so that the driving current varies according to said high-frequency signal, and the wavelength of said laser light is shifted among a plurality of values.

6. An endoscope system comprising:
an excitation-light emission unit including at least one laser-light source which emits multiaxial-mode laser light as excitation light;
a light guide unit which guides said excitation light to an object, and illuminates the object with the excitation light; and
an image pickup unit which picks up a fluorescence image formed with fluorescence light which is emitted from said object in response to illumination with said excitation light.

7. An endoscope system according to claim 6, wherein said excitation-light emission unit comprises a plurality of laser-light sources each of which emits multiaxial-mode laser light.

8. An endoscope system according to claim 6, wherein said laser-light source is a GaN semiconductor laser element, and said excitation light belongs to a wavelength band within a range of 400 to 420 nm.

9. An endoscope system comprising:
an excitation-light emission unit which emits excitation light;
a light guide unit which guides said excitation light to an object, and illuminates the object with the excitation light; and
an image pickup unit which picks up a fluorescence image formed with fluorescence light which is emitted from said object in response to illumination with said excitation light;
said excitation-light emission unit comprises a plurality of laser-light sources each of which emits single-axial-mode laser light having a wavelength and a phase, and the single-axial-mode laser light emitted from one of the plurality of laser-light sources is different in at least one of the wavelength and the phase from the single-axial-mode laser light emitted from another of the plurality of laser-light sources.

10. An endoscope system according to claim 9, wherein said laser-light source is a GaN semiconductor laser element, and said excitation light belongs to a wavelength band within a range of 400 to 420 nm.

11. An endoscope system comprising:
an excitation-light emission unit which emits excitation light;
a light guide unit which guides said excitation light to an object, and illuminates the object with the excitation light;
an image pickup unit which picks up a fluorescence image formed with fluorescence light which is emitted from said object in response to illumination with said excitation light; and
a vibration unit which vibrates said light guide unit.

12. An endoscope system according to claim 11, wherein said laser-light source is a GaN semiconductor laser element, and said excitation light belongs to a wavelength band within a range of 400 to 420 nm.

13. An endoscope system comprising:
an excitation-light emission unit which emits excitation light;
a light guide unit which guides said excitation light to an object, and illuminates the object with the excitation light; and
an image pickup unit which picks up a fluorescence image formed with fluorescence light which is emitted from said object in response to illumination with said excitation light;
said excitation-light emission unit comprises,
a laser-light source which emits as said excitation light laser light having a wavelength,
a high-frequency-signal output unit which outputs a high-frequency signal, and
a driving-current generation unit which generates a driving current of said laser-light source so that the driving current varies according to said high-frequency signal, and the wavelength of said laser light is shifted among a plurality of values.

14. An endoscope system according to claim 13, wherein said laser-light source is a GaN semiconductor laser element, and said excitation light belongs to a wavelength band within a range of 400 to 420 nm.
